(19) United States
(12) Patent Application Publication
(10) Pub. No.: US 2021/0220443 A1
(43) Pub. Date: Jul. 22, 2021

(54) METHOD AND USE OF CONTROLLING POSTPRANDIAL GLUCOSE LEVELS IN A SUBJECT

(71) Applicant: BIOCON LIMITED, Kamataka (IN)

(72) Inventors: SANDEEP N. ATHALYE, KARNATAKA (IN); ANAND KHEDKAR, KARNATAKA (IN); ALEXAMDER FLEMING, HARPERS FERRY (IN); ALAN CHERRINGTON, NASHVILLE, TN (US); ASHWINI VISHWESWARAMURTHY, KARNATAKA (IN); HAROLD E. LEBOVITZ, STATEN ISLAND, NY (US)

(21) Appl. No.: 15/734,044

(22) PCT Filed: Jun. 17, 2019
(86) PCT No.: PCT/IB2019/055016

§ 371 Date: Dec. 1, 2020

(30) Foreign Application Priority Data
Jun. 18, 2018 (IN) 201841022758

(51) Int. Cl. A61K 38/28; A61K 9/00; A61K 47/54; A61P 3/10

(52) U.S. Cl. CPC A61K 38/28; A61P 3/10; A61K 47/54; A61K 9/0053

(57) ABSTRACT

Disclosed is a method of controlling postprandial blood glucose levels in a subject. The method includes orally administering to a subject an orally administrable oligoethylene glycol conjugate of insulin or an orally administrable insulin fusion protein. Oral administration is done during a time period from about 5 to 25 minutes prior to a meal.

### FIELD OF INVENTION

The invention relates to a method and a use of insulin for controlling postprandial blood glucose levels in a subject, in particular in a patient with Diabetes mellitus such as Diabetes mellitus type 2. Disclosed are furthermore a suitable pre-meal dosing time, between-meal-interval time and a meal composition for an orally administered insulin derivative.

### BACKGROUND OF INVENTION

Diabetes mellitus is a metabolic disorder of the glucose metabolism, generally characterized by high blood sugar levels over a prolonged period of time. Forms of the disease can be mainly associated with insulin resistance to the body cells, or impaired production of insulin by the pancreatic β-cells.

Type 2 diabetes mellitus (T2DM) is a complex metabolic disorder, typically characterized by progressive b-cell failure and increasing difficulty in maintaining glycaemic control. It is associated with the increased risk of microvascular and macrovascular complications.

Insulin administered subcutaneously is absorbed non-physiologically into the systemic circulation with a consequent risk of peripheral hyperinsulinemia, Hypoglycaemia and weight gain. Several pre-clinical studies have assessed the metabolic differences between administration of insulin into the portal vein versus systemic vein or artery. Portal vein infusion of human insulin at the rate of 1.8 pmol/kg/min (a rate ˜25% above basal) was observed to 1) result in a rapid (50% to 60%) decline in endogenous glucose production, 2) not increase arterial plasma insulin levels, 3) have no effect on non-hepatic glucose uptake and 4) have a small delayed effect in inhibiting adipose tissue lipolysis. Peripheral administration at the same infusion rate resulted in 1) increase in plasma insulin levels in the peripheral artery by 2-fold without any effect on portal vein insulin concentrations; 2) 2- to 3-fold increase in non-hepatic glucose uptake, 3) EGP suppression only after several hours and 4) significant inhibition of adipose tissue lipolysis. Additionally, peripheral administration diverted glucose disposal from liver to the muscle and resulted in rapid and more severe hypoglycaemia.

Oral insulin leads to insulinization of the liver through the enteral route and is transported through the portal circulation resulting in higher hepatic insulin levels similar to endogenous insulin secretion. The short duration of prandial action and primary portal delivery of oral insulins is expected to provide several clinical advantages, such as: 1) lower incidence of Hypoglycaemia (including nocturnal Hypoglycaemia) compared to the current parenteral routes of insulin administration; 2) lower peripheral hyperinsulinemia; 3) metabolic effects with minimal weight gain; 4) improvement in patient related outcomes (quality of life) and improvement in compliance levels significantly, thereby encouraging early insulinization in patients with possible resultant beta cell sparing. Most oral insulins in the preliminary phases of development were discontinued due to either high variability or lack of absorption in the presence of a meal.

Polymeric nanoparticles can protect orally administered compounds from degradation; and there have been attempts to use insulin-polymer nanoparticles for oral delivery. Vitamin B-12 has also been investigated for conjugation to insulin, and been shown to improve oral bioavailability. Conjugates of insulin and transferrin have been shown to improve drug transport in a mucus-producing co-culture model.

IN-105 (International non-proprietary name: insulin tregopil), a PEGylated recombinant human insulin (100% sequence identity to human insulin), is currently in development for oral delivery in the treatment of diabetes mellitus. It contains a single methoxy-triethylene-glycol-propionyl unit attached to the Lys-B29-amino group of human insulin via an amide linkage. IN-105 is resistant to degradation in the gastrointestinal tract and improves the availability of the intact insulin peptide for intestinal absorption facilitated further by sodium caprate, a functional excipient in the formulation. This feature of IN-105 along with its rapid onset of action and ultra-short action profile, distinguishes it from some other oral insulins It has been demonstrated to be safe and pharmacodynamically active in normal healthy volunteers as well as in patients with Diabetes mellitus type 2.

Plasma levels of peripherally (subcutaneously) injected rapid acting insulins generally peak at 30-60 minutes after injection and have a duration of action up to 3-5 hours. The effect of pre meal dosing time of injected insulin on post prandial glucose (PPG) levels, and the extended effect on overall glycaemic control, is well established. IN-105 is rapidly absorbed (within 30 minutes after dosing) and may restore the first phase insulin release deficiency in patients with Diabetes mellitus type 2. The duration for which prandial insulin effect is required for tight targeted glycemic control is variable and depends on multiple factors such as meal composition and gastric emptying time. It would thus be advantageous to have available a suitable dosing schedule for this rapidly acting IN-105.

Furthermore, there is a need for a method of treating diabetes mellitus, which efficiently controls glucose levels without causing extensive postprandial (post-meal) hypoglycaemia.

### Object of Invention

The main objective of the present invention was to develop a method of controlling postprandial blood glucose levels during a post-meal period in a subject by administering an insulin containing compound.

More particularly, the objective was mainly related to establish pre-meal dosing time for IN-105 administration. The secondary objective was to assess the safety and tolerability of IN-105 administered under different dosing conditions.

### SUMMARY OF THE DISCLOSURE

Disclosed herein is a method of administering an insulin containing compound to a subject. The method is generally a method of administering an orally administrable insulin containing compound. The insulin containing compound typically contains insulin covalently linked to one or more additional moieties. The insulin containing compound may for instance be a fusion protein containing insulin and a further polypeptide, covalently or non-covalently coupled to an insulin chain. The further polypeptide may be covalently or non-covalently coupled to the insulin A chain. The further polypeptide may be covalently or non-covalently coupled to the insulin B chain. The insulin containing compound typically contains insulin, of which one or both chains are covalently linked to one or more additional moieties. In some embodiments the insulin containing compound is a fusion polypeptide, it may for instance be a fusion protein containing insulin and a further polypeptide. In some embodiments the insulin containing compound is a fusion polypeptide that consists of insulin and a further polypeptide such as transferrin.

In some embodiments the insulin containing compound is a fusion polypeptide that consists of insulin and a further polypeptide, wherein at least one of the insulin moiety and the further polypeptide is PEGylated.

In some embodiments the subject is suffering from Diabetes mellitus type 2. In some embodiments the subject is already on anti-diabetic medication, receiving an antidiabetic, such as a biguanide, e.g. metformin, a thiazolidinedione, e.g. rosiglitazone or pioglitazone, or long acting insulins e.g. insulin glargine etc.

Disclosed herein is also a method of controlling postprandial blood glucose levels in a subject. Such a method, as well as the above method of administering an insulin containing compound, may also be a method of reducing postprandial hypoglycaemia during a post-meal period.

Disclosed herein is also an insulin containing compound for use in a method of controlling postprandial blood glucose levels in a subject. Thereby there is also provided the use of an insulin containing compound in the manufacture of a medicament for controlling postprandial blood glucose levels in a subject.

In this regard a method and use disclosed herein can also be taken to define a method and a use of/in treating Diabetes mellitus type 2.

The insulin containing compound may be an oligoethylene glycol conjugate of insulin or an insulin fusion protein. In some embodiments the oligoethylene glycol conjugate of insulin consists of insulin and an oligoethylene glycol moiety, the latter being covalently bonded to the B chain of insulin. The covalent bond may be a non-hydrolysable amide bond. In some embodiments the oligoethylene glycol moiety is bonded to the free amino group on the Lys-b29 residue of insulin. The oligoethylene glycol moiety may in some embodiments be of the following structure:

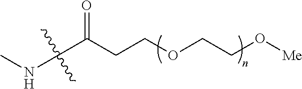

In this structure an amide bond is formed with an amino group of an insulin chain, e.g. lysine at position 29 of the B chain of insulin. The fact that the amino nitrogen is part of the insulin molecule is illustrated by a sidled line. In the above structure n may be an integer from 1 to 6, such as 2 or 4. n may also be the integer 5 or the integer 3.

In some embodiments the insulin containing compound may be represented as follows:

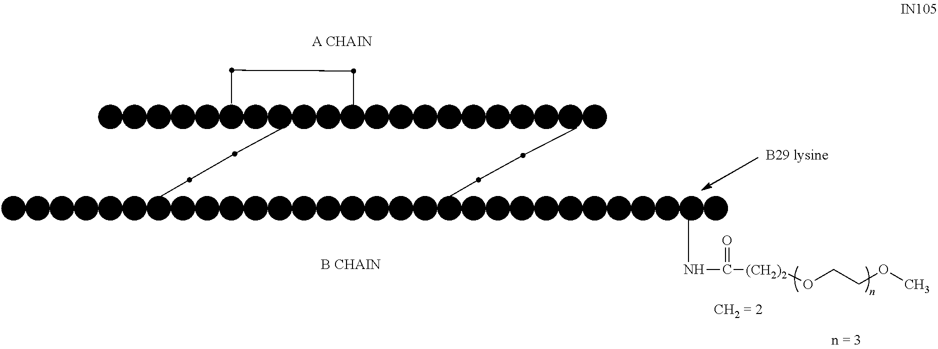

In some embodiments the insulin containing compound is IN-105.

The insulin moiety is typically a mammalian insulin molecule, such as a bovine or a porcine insulin molecule. In some embodiments the insulin moiety is human insulin. In some embodiments the insulin moiety is recombinant insulin. As an illustrative example, the insulin moiety may be recombinant human insulin.

In a method or a use disclosed herein the insulin containing compound is administered to the subject within a certain time frame prior to a meal. In some embodiments the insulin containing compound is administered during a time period from about 5 to about 20 minutes prior to a meal.

### BRIEF DESCRIPTION OF DRAWINGS

FIG. 1 represents study design of Cohort-1, Cohort 2 and Cohort 3.

FIG. 2A represents mean plasma concentration vs time profiles in Cohort-1.

FIG. 2B represents ratio of baseline corrected plasma glucose concentration vs time profiles in Cohort-1.

FIG. 3A represents mean plasma concentration of IN-105 vs time profiles for afternoon meal group in Cohort-2.

FIG. 3B represents mean baseline corrected plasma glucose concentration vs time profiles as difference between post meal glucose and baseline glucose (morning meal) in Cohort-2.

FIG. 3C represents mean baseline corrected plasma glucose concentration vs time profiles as difference between post meal glucose and baseline glucose (afternoon meal) in Cohort-2.

FIG. 4A represents mean plasma concentration vs time profiles in Cohort-3: American Diabetes Association (ADA) meal-ADA meal.

FIG. 4B represents mean plasma concentration vs time profiles in Cohort-3: High fat meal-ADA meal.

FIG. 4C represents mean plasma concentration vs time profiles in Cohort-3: High fat meal-ADA meal.

FIG. 4D represents mean baseline corrected plasma glucose concentration vs time profiles as difference between post meal glucose and baseline glucose (morning meal) in Cohort-3.

FIG. 4E represents mean baseline corrected plasma glucose concentration vs time profiles as difference between post meal glucose and baseline glucose (afternoon meal) in Cohort-3.

### DETAILED DESCRIPTION OF INVENTION

The term “orally administering” as used herein in the context of administering an insulin compound or conjugate includes allowing the subject to orally consume the insulin compound or conjugate.

The term “postprandial” refers to the post-meal period, i.e. the time period after consuming any nutritional or nutraceutical composition as known in the nutritional and nutraceutical arts.

The term “control” as used herein may refer to balancing, managing, stabilizing, modulating, and/or otherwise regulating a biological characteristic, such as the blood sugar level, in a beneficial manner. Controlling the blood sugar level, including the blood glucose level, typically includes reducing the blood glucose level in a time period of about 20 minutes to about 2.5 or 3 hours, in particular 30 or 40 to 120 minutes, following a meal, when compared to the absence of an administered compound. Controlling the blood sugar level or blood glucose level as used herein generally also includes reducing a rise in blood glucose—hence, decreasing blood glucose—in a time period of about 5 minutes to about 30 minutes, in particular about 10 to about 20 minutes, following a meal, when compared to a different administration regime of a compound.

The term “ADA diet” aka “American Diabetes Association Diet” as used herein in the context of diet with fixed caloric and glycaemic index provided to the patients based on recommendation of a registered dietician before the study start.

The term “high fiber diet” as used herein refers to a diet with fixed caloric and glycaemic index containing high amount of dietary fiber approved by FDA and mentioned on its website https://www.accessdata.fda.gov/scripts/interactivenutritionfactsabel/dietarv-fiber.html as accessed on 8 Jun. 2018.

The term “high fat diet” as used herein refers to a diet with fixed caloric and glycaemic index containing high amount of dietary fat approved by FDA and mentioned on its website https://www.accessdata.fda.gov/scripts/lnteractiveSMutritionFactsLabel/fat.html as accessed on 8 Jun. 2018.

The term “excessive” refers to a deviation, whether as an increase or a decrease, which is more than a generally accepted or desired amount.

In a method or use disclosed herein an orally administrable oligoethylene glycol conjugate of insulin or an orally administrable insulin fusion protein is used. The insulin moiety within the oligoethylene glycol conjugate or within the insulin fusion protein may in some embodiments match the species of the subject. In some embodiments the insulin moiety may have an insulin chain of a species that differs from the species of the subject. The insulin moiety may for instance be the human molecule of isoform 1 of chain A of amino acid positions 90-110 and chain B of amino acid positions 25-54 according to Uniprot/Swissprot accession no. P01308, version 1 of the sequence as entered on 21 Jul. 1986. The insulin moiety may for instance be the human molecule of two chains A and B according to VAR_003971 or variant VAR_003974 of Uniprot/Swissprot accession no. P01308, version 237 of the entry of 23 May 2018. The insulin moiety may also be the human molecule of two chains A and B according to variant VAR_003975 of Uniprot/Swissprot accession no. P01308, version 237 of the entry of 23 May 2018. The insulin moiety may also be the human molecule of isoform 2 according to Uniprot/Swissprot accession no. F8WCM5, version 1 of the sequence as entered on 26 Jun. 2013.

The insulin moiety may in some embodiments be the porcine molecule of chain A of amino acid positions 88-108 and chain B of amino acid positions 25-54 according to Uniprot/Swissprot accession no. P01315, version 2 of the sequence as entered on 21 Jul. 1986.

The subject may be a mammal. In some embodiments the subject is a mouse or a rat. The subject may in some embodiments be an ape or a monkey. The subject may also be a human.

Methods and uses disclosed herein achieve a control of the blood glucose level in a subject such as a patient after the subject had a meal. A control of the blood glucose level has been found regardless of the type of meal, e.g. regardless of whether e.g. a high-fat or a high-fiber composition meal was taken be the subject.

The subject is typically suffering from diabetes, for example diabetes mellitus type 2, where insufficient insulin and/or insulin resistance is present. After a meal, especially a high-carbohydrate meal, glucose levels increase. Insulin reduces glucose levels in healthy individuals, while in individuals with diabetes mellitus, the action of insulin is reduced. As a result, larger spikes in blood glucose levels are observed that take longer to return to baseline. Administering insulin or an insulin-containing compound leads to reduction in glucose levels. However, an immediate onset of the reduction of blood glucose levels or continued action of reduction of blood glucose levels beyond the post-meal excursion period, can potentially lead to hypoglycaemia, i.e. a reduction of blood glucose levels below normal values.

Methods and uses disclosed herein are effective in reducing short-term blood glucose levels following a meal, while avoiding an excessive postprandial hypoglycaemia after an initial post meal period. Put differently, both a short-term drop in blood glucose levels thereby providing post prandial glucose control and post meal hypoglycemia are avoided when carrying out a method disclosed herein or applying a use disclosed herein.

As indicated above, the insulin containing compound may be an orally administrable insulin containing compound. In some embodiments oral administration of the orally administrable insulin containing compound is carried out during a time period from about 5 to 25 minutes before a meal. In some embodiments oral administration of the orally administrable insulin containing compound is carried out during a time period from about 10 to 25 minutes before a meal. In some embodiments oral administration of the orally administrable insulin containing compound is carried out during a time period from about 15 to 25 minutes before a meal. In some embodiments oral administration of the orally administrable insulin containing compound is carried out during a time period from about 15 to 20 minutes before a meal. In some embodiments oral administration of the orally administrable insulin containing compound is carried out during a time period from about 5 to 15 minutes or 10 to 15 minutes before a meal.

The insulin containing compound, such as the oligoethylene glycol conjugate or the insulin fusion protein, may be administered several times, such as twice or three times during a selected time interval prior to a meal. In some embodiments the insulin containing compound is administered in a single dose during a selected time interval prior to a meal.

The insulin oligoethylene glycol conjugate is administered at a dose from 10 to 60 mg per individual, such as 15 to 45 mg per individual. In some embodiments 20 mg insulin oligoethylene glycol conjugate are being administered per individual, or 30 mg insulin oligoethylene glycol conjugate. The insulin fusion protein is administered at a dose from 20 to 100 mg per individual, such as 30 to 80 mg per individual. In some embodiments 50 mg insulin fusion protein are being administered per individual, or 60 mg insulin fusion protein.

In case several administrations are carried out the total dose of the oligoethylene glycol conjugate or the insulin fusion protein should amount to a corresponding value.

The oligoethylene glycol conjugate or the insulin fusion protein may be orally administered within a time period as defined above, e.g. from about 7 to 27 minutes, prior to each meal consumed by the subject. In some embodiments the oligoethylene glycol conjugate or the insulin fusion protein may be orally administered within a time period as defined above prior to each main meal, e.g. breakfast, lunch and supper/dinner consumed by the subject. In some embodiments the subject is allowed to have main meals, prior to which the oligoethylene glycol conjugate or the insulin fusion protein is administered, with predefined intermediate intervals between these main meals.

In some embodiments the subject is only allowed to have meals, prior to which the oligoethylene glycol conjugate or the insulin fusion protein is administered, with predefined intermediate intervals between the meals.

The oligoethylene glycol conjugate or the insulin fusion protein may be used as anti-diabetic medication. In some embodiments the oligoethylene glycol conjugate or the insulin fusion protein are the only anti-diabetic medication administered to the subject. In some embodiments anti-diabetic therapy using the oligoethylene glycol conjugate or the insulin fusion protein may be combined with long acting insulin and analogues such as NPH insulin, Glargine, Detemir or Degludec or one or more further oral anti-diabetics, such as a biguanide, e.g. metformin, a thiazolidinedione, e.g. rosiglitazone or pioglitazone, or a Lyn kinase activator, e.g. tolimidone. The administration of such a further anti-diabetic may be independent of the administration of the insulin oligoethylene glycol conjugate or the insulin fusion protein.

The following are examples illustrate the methods and uses disclosed herein. It is understood that various other embodiments may be practiced, given the general description provided above.

### Examples

A Phase I study presented in these Examples was run as a randomized, open-label, placebo controlled, crossover trial, conducted in three cohorts of patients with type-2 diabetes mellitus (T2DM) between March 2014 and July 2014 at a single location in the United States of America. A total of 51 patients with T2DM (24 males: 27 females) between 39-64 years of age were enrolled. Of these, 45 (88.2%) patients were White and 6 (11.8%) patients were Black/African American.

Key inclusion criteria were:

1. Male and female patients from 18 to 65 years, both ages inclusive.
2. An established diagnosis of T2DM per ADA 2013 criteria for at least 1 year prior to screening and are on metformin treatment for at least a month before screening.
3. Body mass index (BMI) of 18.5 to 40.00 kg/m², both inclusive.
4. Stable weight with no more than 5 kg gain or loss in the 3 months prior to screening.
5. Glycosylated hemoglobin (HbAlc)<9.5%.
6. Hemoglobin >9.0 g/dL.
7. Fasting plasma glucose levels less than 140 mg/dL at screening.
8. No clinically significant abnormality in the ECG at screening.
9. The capability of communicating appropriately with the investigator.
10. At screening and baseline, vital signs should be within the following ranges:
a. Oral body temperature between 35.0° C. to 37.5° C.
b. Systolic blood pressure: <140 mm Hg.
c. Diastolic blood pressure: <90 mm Hg.
d. Pulse rate: 50-90 bpm.
e. no clinical manifestations of postural hypotention (no more than a 20 mm Hg drop in systolic or 10 mm Hg drop in diastolic blood pressure)
   11. A written and signed informed consent before starting any protocol-specific procedures.

Key exclusion criteria were:

1. History of hypersensitivity to insulins or insulin analogues.

### 2. Pregnancy

3. Evidence of the hypoglycaemia, limb amputation, diabetic food cancer, diabetic ulcer, severe form of neuropathy, cardiac autonomic neuropathy (either due to improper diabetes control or due to secondary complications following diabetes).
4. Presence of any of human immunodeficiency virus (HIV), hepatitis B (HBsAg) or hepatitis C infection, clinically significant abnormality, Impaired hepatic function, clinically significant chronic renal disease (e.g. nephrotic syndrome, diabetic nephropathy)
5. History or use of OADs other than metformin, oral, intravenous, or inhaled glucocorticoid therapy, prescription drugs, another investigational drug.
6. History of drug or alcohol dependence or abuse; any clinically significant medical conditions such as allergic drug reactions, Autoimmune disorders, Endocrine disorders, Cardiac disease (unstable angina, myocardial infarction); Hematological disorders (e.g. hemoglobinopathies, hemolytic anemia, sickle cell anemia); Neurological disorders (e.g. seizure disorder, stroke, transient ischemic attack); Psychiatric disorders, bipolar affective disorder, schizophrenia); Respiratory disorders; Active cancer; any bleeding or coagulation disorders; Surgical history such as Inflammatory bowel disease, ulcers, gastrointestinal or rectal bleeding, Major gastrointestinal tract surgery such as gastrectomy, cholecystectomy, gastroenterostomy, or bowel resection, Pancreatic injury or pancreatitis
7. Smokers (use of tobacco products in the previous 45 days). Urine cotinine levels will be measured during screening for all patients. Smokers will be defined as patients who report tobacco use and/or who have a urine cotinine level which exceeds 200 ng/ml.
8. Donation or loss of 400 mL or more of blood (or equivalent plasma or blood constituents) within eight (8) weeks prior to initial dosing.

The study was designed, implemented, and reported in accordance with the ICH Harmonized Tripartite Guidelines for Good Clinical Practice (GCP; ICH-E6), with applicable local regulations and ethical principles laid down in Declaration of Helsinki. An independent ethics committee reviewed and approved the protocol and applicable amendments, patient recruitment procedures, and other required documents before study initiation. All patients provided informed written consent prior to enrolment in the study.

### Study Design and Treatments

All 51 patients were divided into 3 cohorts. Total duration of the study participation was approximately 10 weeks for Cohort-1 and 11 weeks each for Cohort-2 and -3.

During recruitment, screening, signing the informed consent and the baseline visits of each treatment period, the patients were informed or reminded of the following restrictions:
No strenuous physical exercise (e.g., weight training, aerobics, football) for 7 days before dosing until after study completion evaluation.
No alcohol for 48 hours before dosing until after last PK blood sample collection.
Intake of xanthine (e.g., caffeine) containing food or beverages must be discontinued 48 hours before dosing until last PK sample collection. Consumption of such foods and beverages (e.g., coffee, tea, caffeine-containing soft drinks, chocolate) was not permitted at any time while the patients are domiciled.
No food other than that decided before the study will be consumed at any time during confinement. When meal and blood draw times coincide, blood was drawn BEFORE the meal was provided.

In all cohorts, patients received IN-105 30 mg (2×15 mg tablets; 240 mL water) or matching placebo. All formulations of metformin that patients were taking prior to study participation were replaced with metformin XR (dose was determined by the investigator based on prior metformin dose taken) at least the previous day of study drug dosing.

Patients from one cohort were eligible to participate in the subsequent cohort if they satisfied the selection criteria (fresh randomization number were assigned after re-screening).

Following the first cohort, evaluation of each successive cohort was modified on the basis of preceding data to address questions sequentially.

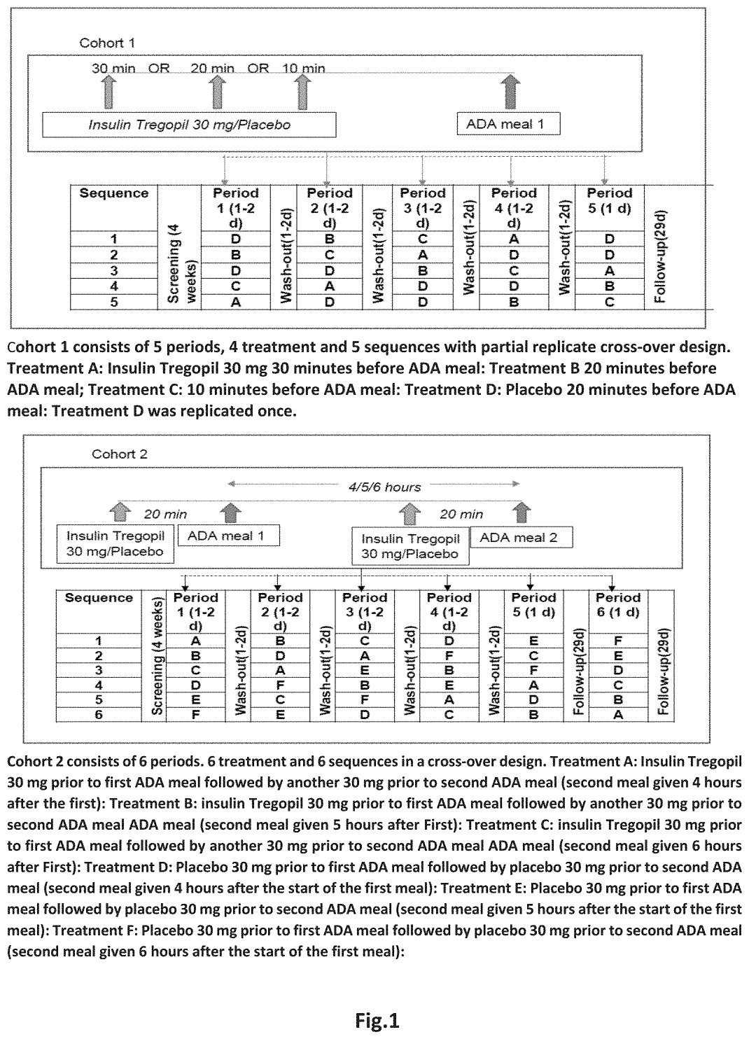

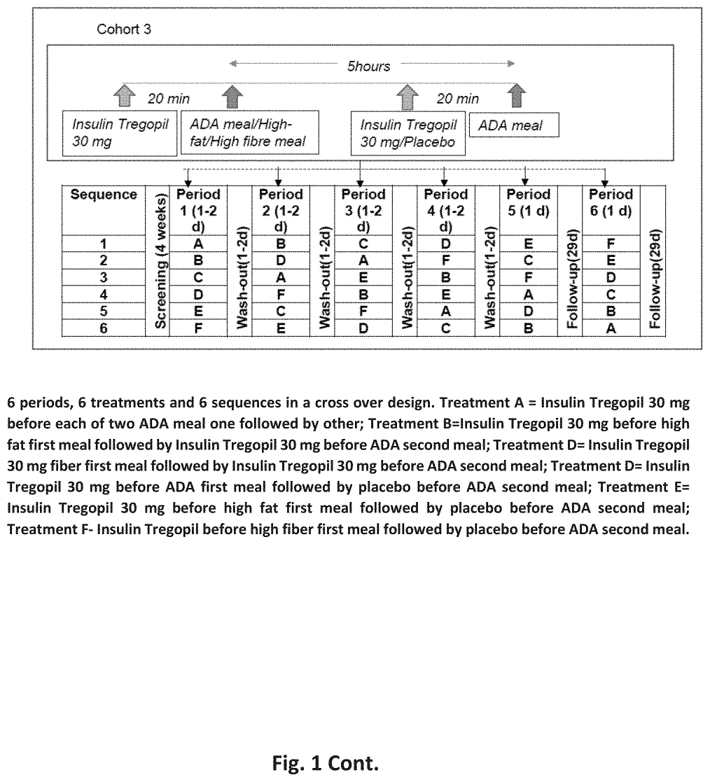

Cohort-1 consisting of 15 patients had a partial replicate crossover design (5 periods/4 treatments (2 weeks)/5 sequences) as shown in FIG. 1A. The study was conducted for about 10 weeks. Washout period between consecutive treatments was 1-2 days. IN-105 was administered 30/20/10 minutes before an American Diabetes Association (ADA)-meal (consumed within 30 minutes) and placebo at 20 minutes before ADA-meal. Placebo was administered twice to each patient to estimate intra-day PD variability.

Cohort-2 consisting of 18 patients had a cross over design (6 periods/6 treatments (3 weeks)/6 sequences) as shown in Figure IB. The study was conducted for about 11 weeks. Washout period between consecutive treatments was 1-2 days. Patients were provided 2 ADA-meals with either IN-105 or placebo administered at the ‘optimum’ pre-meal time selected from Cohort-1 prior to each meal (consumed within 30 minutes). Timing between the meals was maintained at 4, 5, or 6 hours depending on the treatment schedule of each patient.

Cohort-3 consisting of 18 patients had a cross-over design (6 periods/6 treatments (3 weeks)/6 sequences) as shown in FIG. 1C. The study was conducted for about 11 weeks. At the selected pre-meal time (determined from Cohort-1), the patients were administered IN-105 or placebo and were provided 2 sets of meals with an ‘optimum’ between-meal-interval time (determined from Cohort 2). First meal was an ADA/high-fat/high-fiber composition meal while the second meal was an ADA-meal.

Briefly, patients who meet the inclusion/exclusion criteria at screening were enrolled for baseline evaluations. All baseline safety evaluation results were available prior to dosing. Drug was administered −30, −20 and −10 minutes before food intake for cohort 1; at optimal pre-meal time derived from cohort 1 and 4, 5 and 6 hrs after breakfast in cohort 2 and for cohort 3, the drug will be administered at optimal pre-meal time and between-meal interval derived from cohort 1 and 2 respectively.

### Dosage

The design of the study had specific limitations. No active comparators were used in the study as no other oral insulins were available for comparison; all the available ones that could be used as comparators were subcutaneous insulins which were anticipated to have a totally different impact compared to orally delivered insulin mimicking the natural insulin delivery from pancreas. Additionally, a potential treatment dose range of IN-105 was not followed in the study and only a single dose of IN-105 (30 mg) was administered with the meal as the objective was experimental research of optimal administration conductions.

IN-105 was used as 15 mg tablet for oral use, wherein, placebo was used as placebo tablet of 15 mg for oral use.

Metformin used by all the patients was switched to an appropriate dose of metformin XR formulation (Glucophage XR) the previous night of dosing. If metformin was used in the morning by the patient it was switched to evening before the study dosing day to avoid any potential drug interaction. Patients were on XR formulation once daily every night till study completion. Patients were put back on their earlier regular treatment with metformin upon study completion.

### Method of Assessment and Statistical Analysis

The assessment was performed in three ways viz.
evaluation of PK parameters,
evaluation of PD parameters and
safety assessment.

The Pharmacokinetics (PK) samples were analyzed using a validated liquid chromatography-tandem mass spectrometry (LC/MS/MS) for PK estimation with appropriate controls. Blood glucose was measured using a glucose-meter at predefined specific time points during the study. All statistical analyses of the PK and PD parameter estimates were conducted using SAS*Version 9.2.

Pharmacokinetic parameters were calculated using plasma concentration vs. time profile (actual time of sample collection) data of the investigational product in individual patients using Phoenix WinNonlin 6.2 or higher using non-compartmental method. The Cₘₐₓ and Tₘₐₓ were obtained from the concentration time profile data. The Kₑᵢ was estimated by linear regression of the terminal part of the log-concentration-time curve. The AUC₀₋ₜ was determined by the linear trapezoidal rule. The AUCo₋₀₀ was calculated by taking the sum of AUCo₋ₜ and the ratio of last measurable concentration to K_{e|}. The t1/2 was calculated as 0.693/K_{e|}. All concentration values below the limit of quantification (LOQ) were set to “zero” for all PK and statistical calculations. Any missing sample was reported as missing (“M”) and not included for PK and statistical analysis.

Primary PK and PD parameters were represented using descriptive statistics. All other PK and PD parameters were summarized as summary statistics (arithmetic mean, standard deviation [SD], minimum, maximum, median, range, coefficient of variation, standard error, and geometric mean) for all treatments.

No statistical hypothesis was defined for the estimation of the sample size as this was an exploratory study. A minimum of 15 patients in Cohort-1, and 18 patients, each, in Cohort-2 and 3, were deemed adequate for evaluation of the objectives.

The PK and PD population was defined as all randomized patients who received IN-105/placebo and had evaluable data for PK/PD endpoints. For all 3 Cohorts, plasma concentration data for each patient and treatment were analyzed by a non-compartmental method. The AUC₀₋ₜ was calculated by the trapezoidal rule; concentration values below the limit of quantification were set to “zero”. All primary PK and PD parameters were represented using descriptive statistics. All other PK and PD parameters were summarized using summary statistics for all the treatments. Ratios and 90% confidence intervals (Cls) of geometric mean were calculated for PK and PD parameters from mixed effects model with sequence, period and treatment as covariates; and patient-within-sequence as a random effect for log-transformed Cₘₐₓ, and AUC. In Cohort-1, for IN-105, the ratio between AUC_{pOSt}-mₑₐᵢ (plasma concentration from the time of start of meal [10, 20 and 30 minutes post-dose] to the last time point with measurable concentration) and AUCₚᵣₑ-mₑₐᵢ plasma concentration from time of IN-105 administration [10, 20 and 30 minutes prior to meal] to the time of meal) is represented as percentage.

The PD response in Cohort-2 following the morning dose of IN-105 (A, B and C groups) and placebo (D, E and F groups) were used for IN-105 and placebo-related variability calculations, respectively. Plasma glucose level at time zero was the PD baseline.

Safety was evaluated in all randomized patients who received at least one dose of the study drug and were analyzed according to the treatment sequence using descriptive statistics. Adverse events were coded using MedDRA*, version 17.0. All available safety data were collected until the end of the study.

The PK parameters evaluated were as follows:
Cₘₐₓ (observed maximum plasma concentration following single dose drug administration),
AUQ_{H} (area under the plasma-concentration time curve up to the last measured concentration at time t) and
Tₘₐₓ (time required to achieve maximum plasma concentration).

The PD parameters evaluated were as follows:
AUCiₐₛₜ (area under the plasma concentration time curve up to the last quantifiable concentration),
tₘᵢₙ (time to minimum glucose concentration) and
Cₘᵢₙ (minimum glucose concentration).
The Safety assessments evaluated were as follows:
physical examinations,
electrocardiogram (ECGs),
vital signs,
clinical laboratory evaluations (including hematology, blood chemistry and urinalysis),
blood glucose by glucose meter
treatment-emergent adverse event (TEAE) and
serious adverse event (SAE) monitoring.

IN-105 given orally 10-20 minutes before major meals, was rapidly absorbed attaining adequate post-meal exposure and was effective in lowering PPG excursions when meals were separated by about 5 hours. Furthermore, the efficacy of IN-105 was not altered by meal type as demonstrated by its glucose-lowering response in the post meal period.

Overall, IN-105 administered under various dosing conditions and with different meal types was safe and well tolerated in patients with T2DM.

### Example 1: Cohort-1 Study

Cohort-1 had a partial replicate crossover design (5 periods/4 treatments (2 weeks)/5 sequences) as shown in FIG. 1A. A total of 15 patients were enrolled. IN-105 was administered at 30, 20 and 10 minutes before the meal. Placebo was administered only 20 minutes before the meal. To estimate the pharmacodynamic (blood glucose level) variability on different days, placebo was administered twice to every patient. Cohort 1 consisted of 5 periods, 4 treatments and 5 sequences with a partial replicate crossover design as shown in below table 1 and FIG. 1. Three patients were randomly assigned to each of the 5 treatment sequences. Each patient went through the 4 treatments, A through D, in a cross-over fashion. The washout period between the treatments was a minimum of 1 day to a maximum of 2 days. The dose of IN-105 was 30 mg (2×15 mg tablets) or matching placebo administered with 240 mL of water. An ADA (American Diabetes Association) recommended diet was consumed. All patients consumed the meal completely within 30 minutes. The study design was as shown in table 1 and FIG. 1.

TABLE 1
Treatment details for Cohort 1

| Name | Description |
|---|---|
| Treatment A | IN-105 30 mg 30 min before ADA meal |
| Treatment B | IN-105 30 mg 20 min before ADA meal |
| Treatment C | IN-105 30 mg 10 min before ADA meal |
| Treatment D (twice each patient) | Placebo 20 min before ADA meal |

2.5 mL of blood was collected after meal administration at the time points such as—0 hours, 10, 20, 30, 40, 50, 60, 90, 120 and 180 minutes post-meal. In addition, blood samples were collected at −30, −20, −10 minutes in relation to the meal (pre-meal). The zero hour time point for cohort 1 will be in relation to the start of the meal such that time zero will be at the start of the meal. IN-105 PK and plasma glucose levels were measured for all the samples.

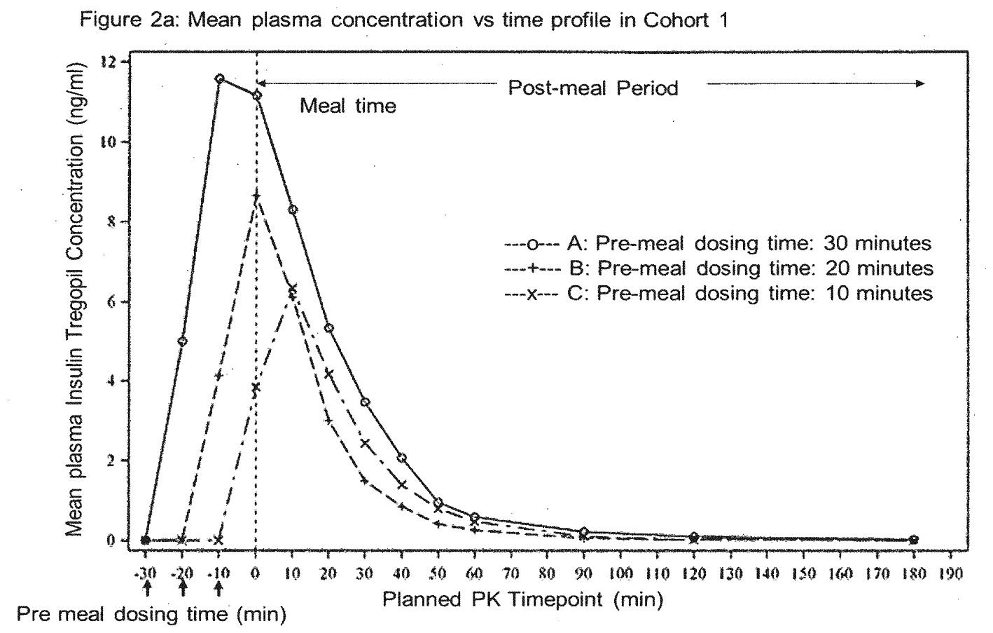

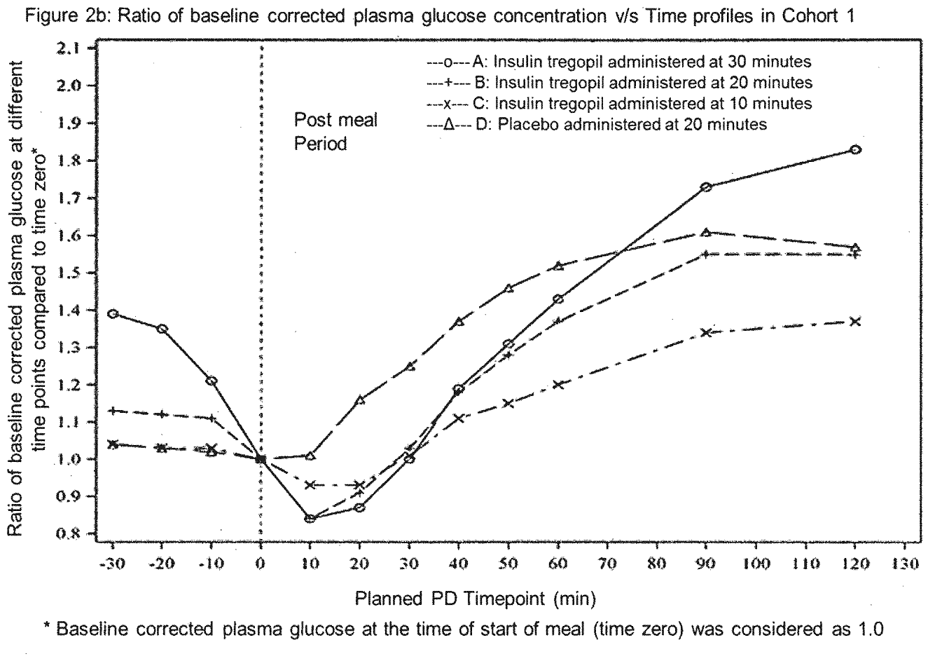

Mean IN-105 plasma concentrations as a function of the pre-meal time of closing and the effect on the corresponding plasma glucose levels (PD) are shown in FIG. 2A and FIG. 2B. The PK and PD parameters in terms of AUC₀-isoₘᵢₙ, Cₘᵢₙ, tₘᵢₙ for Cohort-1 are summarized in table 2.

TABLE 2
Pharmacodynamic Parameters for Plasma IN-105 in Cohort-1

| | PD Parameters | | |
|---|---|---|---|
| | Baseline-Corrected Arithmetic Mean | | |
| | Plasma Glucose (Relative to Time Zero) | | |
| | AUC₀₋₁₈₀ | Cₘᵢₙ | Tₘᵢₙ |
| Treatment groups | (min*mg/mL) ± SD | (mg/mL) ± SD | (min) ± SD |
| IN-105 administered 30 | 217.39 ± 31.58 | 0.86 ± 0.11 | 37.33 ± 59.09 |
| minutes before meal | | | |
| IN-105 administered 20 | 227.04 ± 67.89 | 0.81 ± 0.10 | 26.00 ± 43.22 |
| minutes before meal | | | |
| IN-105 administered 10 | 267.67 ± 46.81 | 0.81 ± 0.08 | 16.66 ± 7.23 |
| minutes before meal | | | |
| Placebo administered 20 | 251.96 ± 39.11 | 0.98 ± 0.03 | 33.66 ± 66.72 |
| minutes before meal | | | |

AUC₀₋₁₈₀ area under the curve from time zero to 180 minutes;
Cₘᵢₙ—minimum glucose concentration;,
tₘᵢₙ—time to minimum glucose concentration;
SD—Standard deviation

For PK evaluation, Treatment A was compared with Treatments B and C using appropriated statistical model. For PD evaluation, Treatment D was compared with Treatments A, B, and C using appropriate statistical model. Intra- and inter-patient PD variability for Treatment D was also calculated (primary PK parameters included; AUC₀₋ₜ, Cₘₐₓ, and PD parameters included AUCo₋ₜ[AUC both above and below the baseline values], Cₘᵢₙ and Tₘᵢₙ). For PK, the AUC assessment comparison starting point was drug administration time, and for PD, the AUC assessment comparison starting point was the food administration time.

### Pharmacokinetics Results of Cohort-1:

When IN-105 was administered 10 and 20 minutes before food administration, the IN-105 absorption (AUC) was 50% and 61% and Cₘₐₓ was 57% and 69%, respectively compared to 30 minute administration. When IN-105 was administered 10, 20 and 30 minutes before food administration, the Tₘₐₓ values were 19, 20 and 25 min, respectively. The respective absorption ratios, after meal to before meal for IN-105, were 10.6, 2.1 and 1.1 at 10, 20 and 30 minutes, respectively. The maximum absorption was noted at 30 minutes. FIG. 2A shows the diagrammatic representation of same.

When compared to the 30 minute pre-meal dosing group, systemic exposure (AUC₀₋ᵢₛₒₘᵢₙ and Cₘₐₓ) of IN-105 was found to be 47% and 57% in the 10-minute pre-meal dosing group; and 57% and 69% in the 20-minute pre-meal dosing group. IN-05 pre-meal dosing time of 10 and 20 minutes resulted in lesser AUC₀₋ᵢₛₒₘᵢₙ (arithmetic mean) of glucose compared to placebo. Geometric Mean ratio (GM ratio) of baseline-corrected plasma glucose values observed for pre meal dosing time of 10, 20 and 30 minutes compared to placebo were 87%, 80%, 106%, respectively, for AUCo-isoₘᵢₙ; and 88%, 83%, and 83%, respectively for Cₘᵢₙ. Evaluation of baseline-corrected PD parameters showed mean AUC₀₋ᵢₛₒₘᵢₙ of glucose was numerically lowest for pre-meal dosing time of 10 minutes followed by 20 minutes (no statistically significant difference observed).

### Pharmacodynamic Results of Cohort-1:

When IN-105 was administered 10, 20 and 30 minutes before food administration, the glucose lowering responses (AUC) noted were 92%, 73% and 83%, respectively compared to placebo; minimum glucose level (Cₘᵢₙ) noted were 93%, 76% and 64%, respectively compared to placebo. Time to minimum glucose concentration (Tₘᵢₙ) was 37, 26 and 17 minutes at 10, 20 and 30 minutes, respectively compared to 34 minutes with placebo. Administration of IN-105 20 minutes before food intake produced the best response. Baseline-adjusted data showed lower response for both AUC (87%, 80% and 106%) and Cₘᵢₙ (88%, 83% and 83%), especially for 30 minutes administration. This could be because the study captured the glucose response from the time of meal and not from the time of drug administration. This led to underestimating the pharmacodynamic response of IN-105, as the lowering of plasma glucose levels from the time of drug administration until the time of meal was not captured.

The following observations were noted for the IN-105 baseline-unadjusted PD parameters AUC_{0-180 min} and Cₘₐₓ
for the 20 minute group, baseline-unadjusted AUC₀-isomin was lower than placebo
for both 30 and 20 minute groups, the baseline-unadjusted Cₘᵢₙ was lower than placebo.
   Therefore, the 20 minute group produced the best response; though the 30 minute group produced lower Cₘᵢₙ and AUC₀-iₛₒₘᵢₙ, there was not a significant change.

### Example 2: Cohort-2 Study

Cohort-2 had a cross over design (6 periods/6 treatments (3 weeks)/6 sequences) as shown in Figure IB. 18 patients were planned to enroll. Three patients were randomly assigned to each of the 6 sequences, A through F. Patients were provided 2 meals with IN-105 administered at the 20 minutes pre-meal time as determined from Cohort 1; the first and second doses were either IN-105 30 mg or placebo. The timing between the meals was 4, 5 or 6 hours, depending on the treatment period. Cohort 2 consisted of 6 periods, 6 treatments and 6 sequences in a cross over design as show in table 3 and FIG. 1. Each patient went through all 6 treatments in a cross-over fashion. The washout period between the treatments was a minimum of 1 day to a maximum of 2 days. IN-105 30 mg or matching placebo was administered with 240 mL of water. An ADA recommended diet was provided and all patients consumed meals completely within 30 minutes. IN-105 PK and plasma glucose levels were to be measured after the first and second dose over 3 hours post-dose for treatments A, B and C.

TABLE 3
Treatment details for Cohort 2

| Name | Description |
|---|---|
| Treatment A | IN-105 30 mg prior to first meal followed |
| | by IN-105 30 mg prior to second meal. |
| | Second ADA meal given 4 hours after |
| | start of first ADA meal. |
| Treatment B | IN-105 30 mg prior to first meal followed |
| | by IN-105 30 mg prior to second meal. |
| | Second ADA meal given 5 hours after |
| | start of first ADA meal. |
| Treatment C | IN-105 30 mg prior to first meal followed |
| | by IN-105 30 mg prior to second meal. |
| | Second ADA meal given 6 hours after |
| | start of first ADA meal. |
| Treatment D | Placebo 30 mg prior to first meal followed |
| | by placebo 30 mg prior to second meal. |
| | Second ADA meal given 4 hours after |
| | start of first ADA meal. |
| Treatment E | Placebo 30 mg prior to first meal followed |
| | by placebo 30 mg prior to second meal. |
| | Second ADA meal given 5 hours after |
| | start of first ADA meal. |
| Treatment F | Placebo 30 mg prior to first meal followed |
| | by placebo 30 mg prior to second meal. |
| | Second ADA meal given 6 hours after |
| | start of ADA meal. |

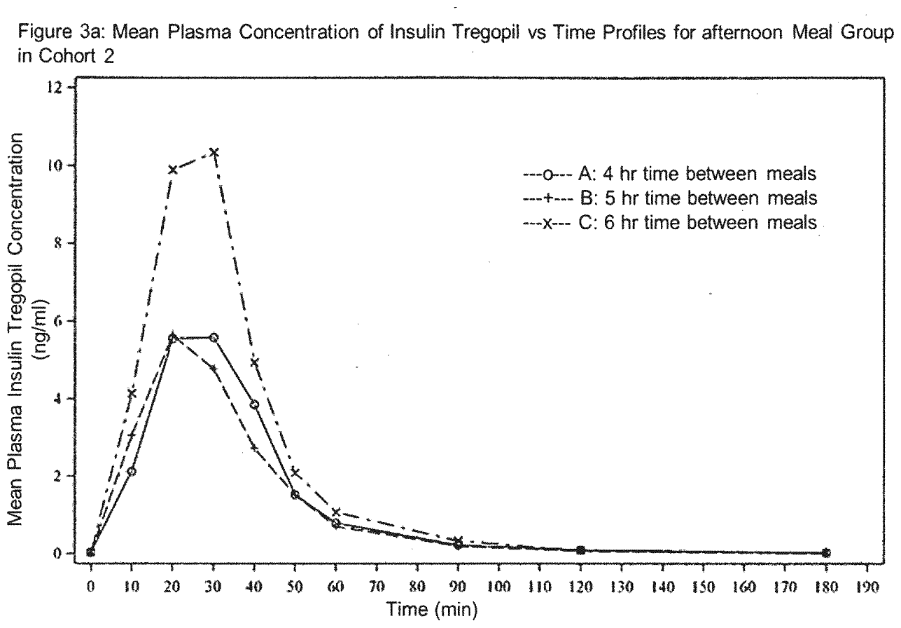

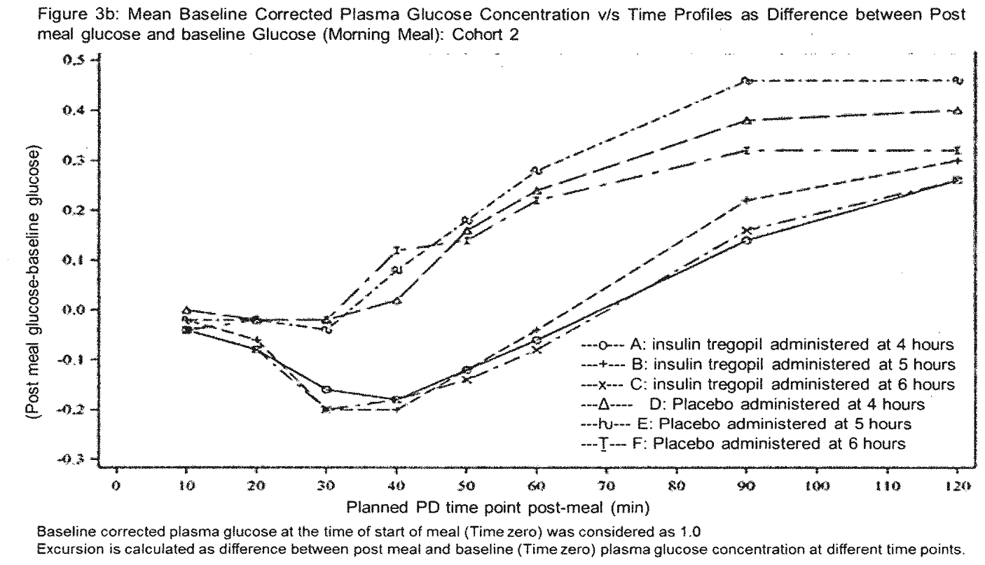

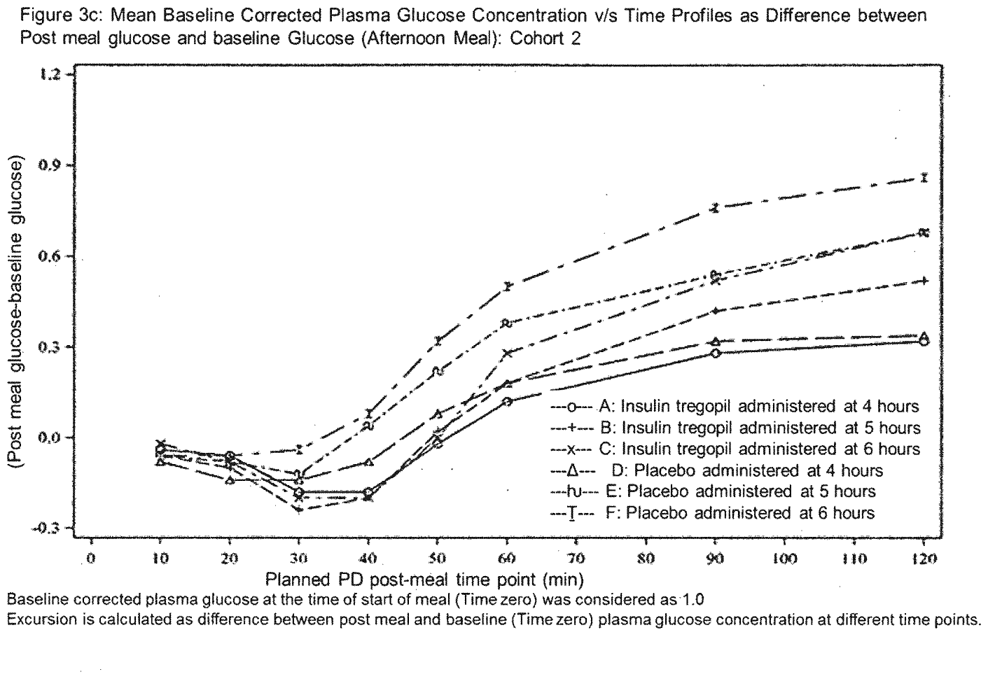

Blood samples (2.5 mL whole blood for PK analysis and 1.5 mL whole blood for PD analysis) were obtained at 0 hour (pre-dose at dosing time), and then at the following time points after IN-105 or placebo administration 10, 20, 30, 40, 50, 60, 90, 120 and 180 minutes post-dose. The following PK parameters were evaluated: Tmax Cmax, AUCO₋ₜ, AUC0₋₀₀, t1/2, Kel, and AUC extrapolated (%) which are summarized in below table 4. Mean IN-105 plasma concentration following the afternoon IN-105 administration at 4, 5 and 6 hours after the morning IN-105 and meal is shown in FIG. 3A. Comparable glucose responses expressed as the difference between post meal glucose and baseline glucose for both the morning and evening meals are shown in FIG. 3B and FIG. 3C. PK and PD parameters for Cohort-2 are summarized in the table 4. For PK and PD evaluation, Treatment C second dose was compared with Treatments A and B second dose using appropriate statistical model. PK and PD were also compared between first and second dose for treatments A, B and C. In addition, PD of Treatment A was compared with treatment D; Treatment B was compared with Treatment E and treatment C was compared with Treatment F. Necessary placebo correction was also applied for PD assessment to eliminate any diurnal effect.

TABLE 4
Pharmacodynamic parameters for Plasma IN-105 and Placebo in Cohort-2

| | | | | |
|---|---|---|---|---|
| Time between | | | | |
| morning and | | AUC₀₋₁₈₀ | Cₘᵢₙ | Tₘᵢₙ |
| afternoon | Time of | (min*mg/dL) ± | (mg/mL) ± | (min) ± |
| ADA meals | meal | SD* | SD* | SD* |

PD parameters for IN-105 [Baseline-Corrected Plasma Glucose (Relative to Time Zero)]

| | | | | |
|---|---|---|---|---|
| 4 hours | Morning | 193.60 ± 27.46 | 0.77 ± 0.08 | 38.88 ± 10.22 |
| | Afternoon | 212.46 ± 33.53 | 0.77 ± 0.18 | 36.16 ± 6.21 |
| 5 hours | Morning | 199.19 ± 23.95 | 0.76 ± 0.09 | 36.11 ± 9.78 |
| | Afternoon | 231.18 ± 54.29 | 0.72 ± 0.17 | 37.33 ± 15.67 |
| 6 hours | Morning | 195.57 ± 26.91 | 0.76 ± 0.11 | 38.88 ± 10.78 |
| | Afternoon | 249.79 ± 39.02 | 0.76 ± 0.12 | 36.66 ± 5.94 |

PD parameters for placebo [Baseline-Corrected Plasma Glucose (Relative to Time Zero)]

| | | | | |
|---|---|---|---|---|
| 4 hours | Morning | 223.90 ± 27.78 | 0.93 ± 0.05 | 23.88 ± 15.00 |
| | Afternoon | 214.14 ± 42.85 | 0.82 ± 0.15 | 40.55 ± 35.55 |
| 5 hours | Morning | 233.77 ± 24.51 | 0.94 ± 0.05 | 18.88 ± 12.78 |
| | Afternoon | 255.55 ± 58.99 | 0.85 ± 0.17 | 23.88 ± 9.78 |
| 6 hours | Morning | 215.95 ± 15.94 | 0.90 ± 0.09 | 34.11 ± 43.31 |
| | Afternoon | 279.83 ± 76.69 | 0.90 ± 0.19 | 10.58 ± 13.90 |

AUCo-iso—area under the curve from time zero to 180 minutes;
Cₘᵢₙ—minimum glucose concentration; ,
tₘᵢₙ—time to minimum glucose concentration;
GMR—geometric mean ratio;
SD—standard deviation
^{#}Baseline-corrected geometric mean values
*Baseline-corrected arithmetic mean values are presented

For the afternoon dose of IN-105, administered 4 and 5 hours after the morning meal, GMRs when compared to 6 hours between-meal-interval for AUCo₋ᵢₛₒₘᵢₙ were 37% and 47%; and Cₘₐₓ were 43% and 57%, respectively. Tₘₐₓ values were 29, 26 and 26 minutes in the morning; and 23, 23 and 27 minutes in the afternoon for the groups with 4, 5 and 6 hours between-meal-interval, respectively.

### Pharmacokinetics Result of Cohort-2:

When IN-105 was administered 4 and 5 hours after morning food intake, the IN-105 AUC was 68% and 76% and Cₘₐₓ was 43% and 57%, respectively compared to 6 hours administration. At 4, 5 and 6 hours after morning food intake, the Tₘₐₓ values were 29, 26 and 26 minutes, compared to 23, 23 and 27 minutes at 4, 5 and 6 hours after afternoon food intake, respectively. The AUC and Cₘₐₓ were lower at noon compared to morning administration. The maximum absorption was noted at 6 hours.

### Pharmacodynamics Results of Cohort-2:

When IN-105 was administered 4, 5 and 6 hours after the previous ADA meal, the AUC values were 117%, 107% and 102%, respectively; and the Cₘᵢₙ values were 112%, 106% and 101% after the second meal, compared to the first meal. The Tₘᵢₙ after 4, 5 and 6 hours after food intake were 36, 37 and 37 minutes, respectively compared to 41, 24 and 11 minutes following placebo, from the time of drug intake respectively.

The following observations were noted for the IN-105 PD parameters, AUC₀₋ᵢₛₒₘᵢₙ and Cₘₐₓ:
For the patients in the 6 hour group, AUC₀₋ᵢₛₒₘᵢₙ was higher in the evening compared to that in the morning (without drug intake)
For the patients in the 4 hour group, AUC₀₋ᵢₛₒₘᵢₙ was lower the evening compared to that in the 6 hour group (after drug intake)
For the patients in the 4 hour group, AUCo-ᵢₛₒₘᵢₙ was lower the evening compared to that in the 6 hour group before drug intake
For the patients in the 4 hour group, Cₘᵢₙ without drug intake was lower in the evening compared to that in the morning
For the patients in the 5 hour and 6 hour groups, Cₘᵢₙ in the evening after drug intake was lower than that in the evening without drug intake

Pooled intra-subject and inter-subject precision (co-efficient of variation, %) for plasma IN-105 PK were as follows: AUC₀₋ᵢₛₒₘᵢₙ inter-subject—144 min*ng/mL, AUC₀-iso intra-subject—81.5 min*ng/mL, Cₘₐₓ inter-subject: 124.7 ng/mL and Cₘₐₓ intra-subject: 64.7 ng/mL. Intra-subject variability for the baseline-adjusted PD response was low for both placebo and IN-105 [variability for AUC₀₋ᵢₛₒₘᵢₙ was 9.17 and 8.41, and for Cₘᵢₙ was 9.16 and 11.61, respectively]. Considering the typical meal interval time and for the convenience of the study subjects, 5 hours was selected as the between-meal interval time for IN-105 dosing in Cohort-3.

The following observations were made for the IN-105 PK parameters AUC₀₋ᵢₛₒₘᵢₙ and Cₘₐₓ:
After the afternoon IN-105 dose, patients in the 4 hour and 5 hour groups had lower AUCo-180ₘᵢₙ and Cₘₐₓ values compared to the 6 hour group. In addition, these values were also lower when compared to their respective morning doses.
Food affects IN-105 absorption for up to 5 hours.

### Example 3: Cohort-3 Study

Cohort-3 had a cross-over design (6 periods/6 treatments (3 weeks)/6 sequences) shown in FIG. 1C. 18 patients were enrolled. Three patients were randomly assigned to each of 6 sequences. The 6 treatments and sequences are depicted below in table 5 and FIG. 1. Patients were randomized to 6 treatment sequences. Patients were provided 2 meals with IN-105 administered at 20 minutes pre-meal time determined from Cohort 1; the first meal was an ADA or high-fat or high-fiber composition meal while the second was an ADA meal provided at 5 hours between meal timing as determined from Cohort 2. All patients consumed meals within 30 minutes. The first dose was IN-105 30 mg while the second dose was either 30 mg of IN-105 or placebo.

This cohort consisted of 6 periods, 6 treatments and 6 sequences in a cross over design. Each patient was administered the 6 treatments in a cross over fashion. The washout period between the treatments was a minimum of 1 day to a maximum of 2 days. The dose of IN-105 was 30 mg (2×15-mg tablets) or matching placebo with 240 mL of water. An ADA recommended diet, or high-fat, or high-fiber diet was consumed.

TABLE 5
Treatment details for Cohort 3

| Name | Description |
|---|---|
| Treatment A | IN-105 30 mg prior to first meal followed |
| | by IN-105 30 mg prior to second meal. |
| | First meal will be an ADA meal; second |
| | meal will also be an ADA meal. |
| Treatment B | IN-105 30 mg prior to first meal followed |
| | by IN-105 30 mg prior to second meal. |
| | First meal will be a high fat meal; |
| | second meal will be an ADA meal. |
| Treatment C | IN-105 30 mg prior to first meal followed |
| | by IN-105 30 mg prior to second meal. |
| | First meal will be a high fiber meal; |
| | second meal will be an ADA meal. |
| Treatment D | IN-105 30 mg prior to first meal followed |
| | by Placebo 30 mg prior to second meal. |
| | First meal will be an ADA meal; |
| | second meal will also be an ADA meal. |
| Treatment E | IN-105 30 mg prior to first meal followed |
| | by Placebo 30 mg prior to second meal. |
| | First meal will be a high fat meal; |
| | second meal will be an ADA meal. |
| Treatment F | IN-105 30 mg prior to first meal followed |
| | by Placebo 30 mg prior to second meal. |
| | First meal will be a high fiber meal; |
| | second meal will be an ADA meal. |

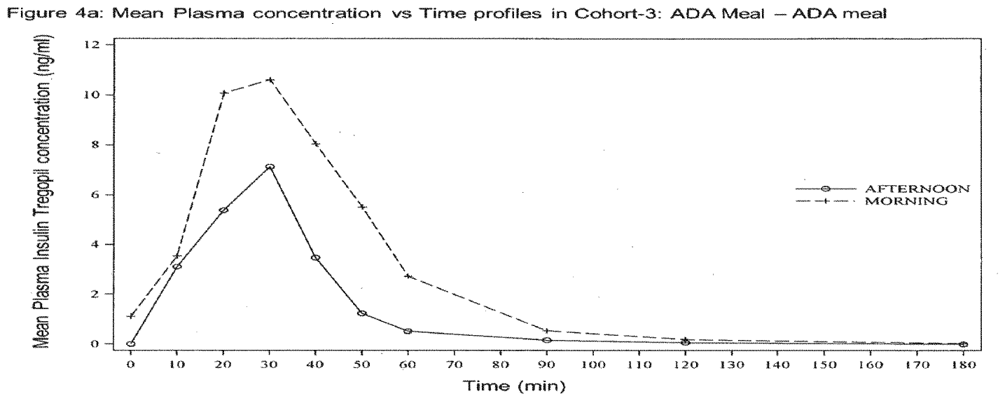

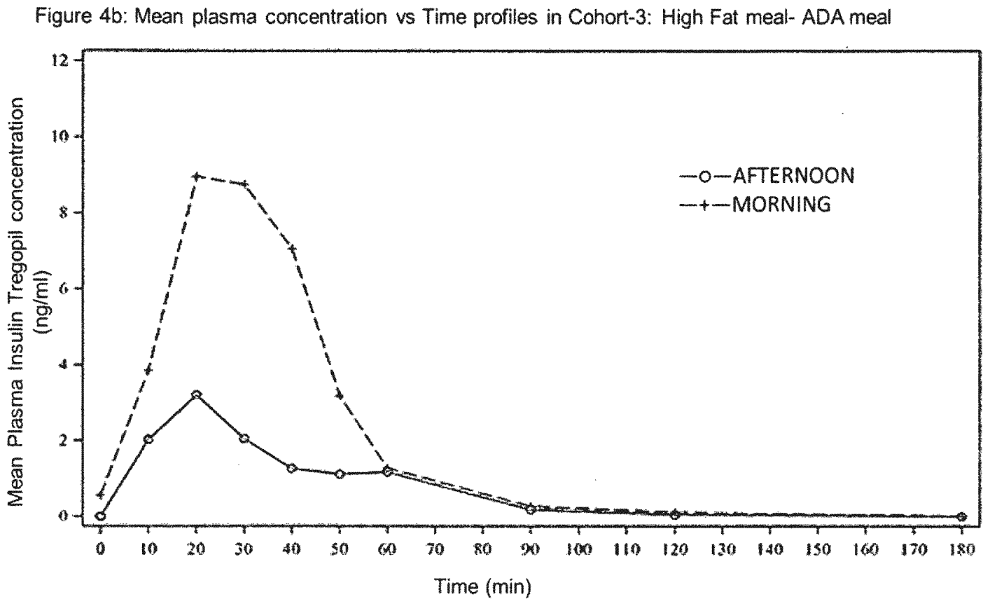

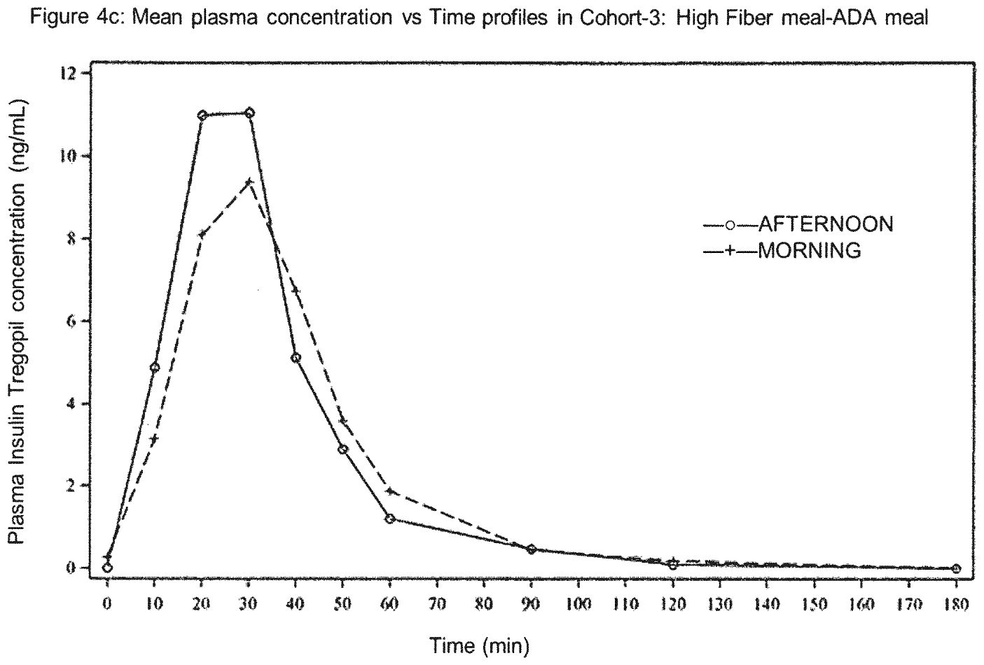

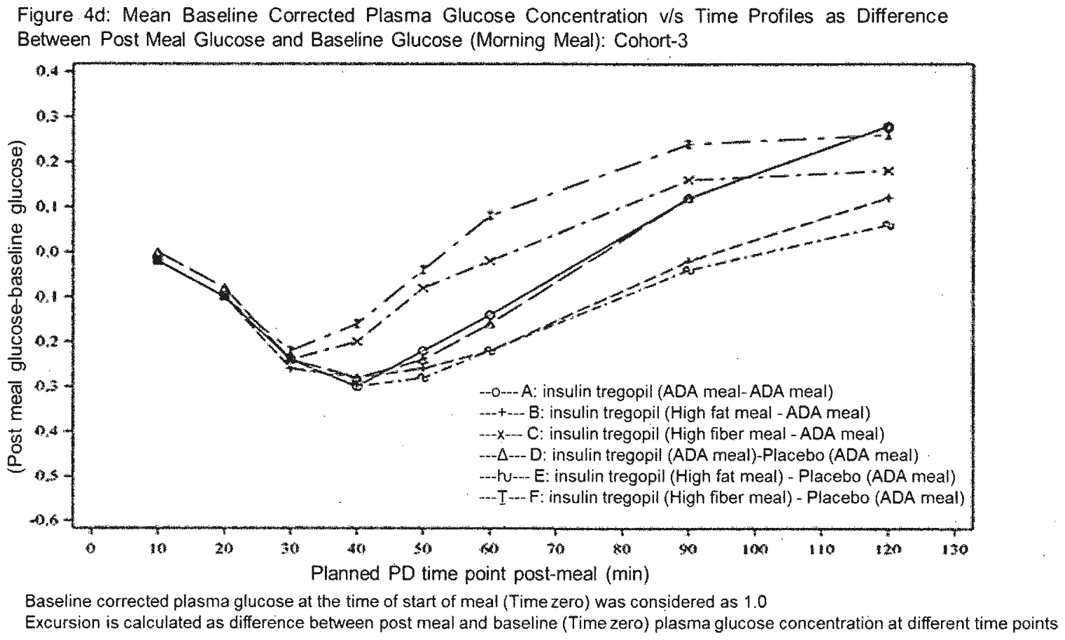

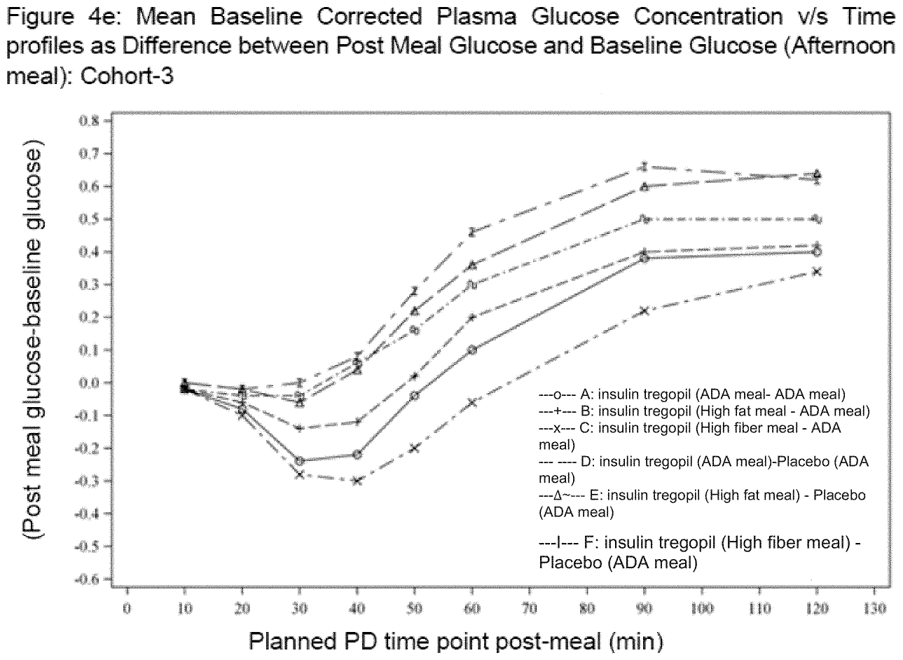

Blood samples (2.5 mL whole blood for PK analysis and 1.5 mL whole blood for PD analysis) were obtained at 0 hour (pre-dose at dosing time), and then at the following time points after IN-105 or placebo administration 10, 20, 30, 40, 50, 60, 90, 120 and 180 minutes post-dose. The following PK parameters were evaluated: Tmax, Cmax, AUCO-t, AUC0-_{∞}, t1/2, Kel, and AUC extrapolated (%) and summarized in below table 6. Mean IN-105 plasma concentrations versus time profiles for ADA-meal-ADA-meal is shown in FIG. 4A, high fat meal-ADA-meal is shown in FIG. 4B and high fiber meal-ADA-meal is shown in FIG. 4C. Comparable glucose responses expressed as the difference between post meal glucose and baseline glucose for both the morning and evening meals are shown in FIG. 4D and FIG. 4E. The PD parameters for Cohort 3 are summarized in Table 6.

TABLE 6
Pharmacodynamic Parameters for Plasma IN-105 and Placebo in Cohort-3
Pharmacokinetics results of Cohort-3
To study the effect of composition of meal on co-administered IN-105,
PK/PD comparison was done between high-fiber (Treatment C first dose)
versus ADA (Treatment A first dose) and high fat (Treatment B first dose)
versus ADA (Treatment A first dose) groups for meal 1. To study the
PK/PD effet of composition of meal on subsequent dose of IN-105,
placebo corrected comparison (for PD) was done between high-fiber
(Treatment C second dose) versus ADA (Treatment A second dose)
and high-fat (Treatment B second dose) versus ADA (Treatment A
second dose) groups for meal 2. Necessary placebo correction was
made for PD assessment to eliminate any diurnal effect.

| | | | | |
|---|---|---|---|---|
| Type of meal, | | AUC₀₋₁₈₀ | Cₘᵢₙ | Tₘᵢₙ |
| morning - | Time of | (min*mg/mL) ± | (mg/mL) ± | (min) ± |
| afternoon | meal | SD* | SD* | SD* |

PD parameters for IN-105

| | | | | |
|---|---|---|---|---|
| ADA - ADA | Morning | 190.68 ± 29.12 | 0.67 ± 0.16 | 40.58 ± 8.99 |
| | Afternoon | 216.13 ± 38.41 | 0.71 ± 0.14 | 38.23 ± 14.67 |
| High-fat - ADA | Morning | 173.81 ± 30.82 | 0.69 ± 0.15 | 42.22 ± 11.66 |
| | Afternoon | 215.28 ± 56.74 | 0.82 ± 0.12 | 37.44 ± 25.29 |
| High-fiber - ADA | Morning | 186.71 ± 30.23 | 0.73 ± 0.15 | 46.47 ± 37.23 |
| | Afternoon | 205.46 ± 37.86 | 0.64 ± 0.13 | 41.17 ± 7.81 |

PD parameters for placebo

| | | | | |
|---|---|---|---|---|
| ADA - ADA | Morning | 191.76 ± 27.20 | 0.70 ± 0.13 | 38.44 ± 13.74 |
| | Afernoon | 256.99 ± 43.38 | 0.91 ± 0.06 | 22.77 ± 14.87 |
| High-fat - ADA | Morning | 169.65 ± 18.63 | 0.66 ± 0.16 | 48.82 ± 20.88 |
| | Afternoon | 238.68 ± 31.76 | 0.92 ± 0.07 | 24.88 ± 14.09 |
| High-fiber - ADA | Morning | 197.49 ± 35.23 | 0.75 ± 0.13 | 40.88 ± 37.67 |
| | Afternoon | 257.13 ± 35.19 | 0.93 ± 0.07 | 19.23 ± 13.27 |

AUCo-iso—area under the curve from time zero to 180 minutes;
Cₘᵢₙ—minimum glucose concentration;
tₘᵢₙ—time to minimum glucose concentration;
GMR—geometric mean ratio;
SD—standard deviation
*Arithmetic mean values are presented
^{#}Geometric Mean values are presented

Morning high fat meal and morning high fiber meal modified IN-105 AUC to 61% and 108% as well as Cₘₐₓ to 63% and 170%, respectively compared to the morning ADA meal. The Tₘₐₓ following morning ADA meal, high-fat meal and high-fiber meal were 25, 25 and 26, respectively compared to 27, 28 and 26 minutes, respectively following the above mentioned afternoon meals. Morning high-fat meal decreased and high-fiber meal increased subsequent IN-105 absorption. (FIG. 4A, 4B, 4C)

### Pharmacodynamics Results of Cohort-3

Morning high-fat meal and morning high-fiber meal modified AUC following IN-105 to 99% and 94% as well as Cmin to 116% and 90%, respectively compared to morning ADA meal. The Tmin following morning ADA meal, high fat meal and high fiber meal were 38, 37 and 41 minutes, respectively. Morning high-fat meal decreased and high-fiber meal increased the glucose lowering response. (FIG. 4D, 4E)

Morning high fat meal and high fiber meal modified IN-105 AUC₀-isoₘᵢₙ to 64.7% and 86.7% as well as Cₘₐₓ to 64.7% and 87.9%, respectively, compared to the morning ADA-meal. Tₘₐₓ of IN-105 was similar in the different meal composition groups (both morning and afternoon dosing). High fiber meal and high-fat meal administration in the morning led to lower plasma glucose AUC₀-i80ₘᵢₙ after morning as well as afternoon dose of IN-105 (placebo corrected levels) compared to the morning ADA-meal. Changes in the glucose AUC_{|ast} and glucose Cₘᵢₙ observed with different types of meals were not consistently significant across the treatment groups despite the observed differences in PK parameters.

The following observations were made for the IN-105 PK parameters AUC₀₋ᵢₛₒₘᵢₙ and Cₘₐₓ:
after the high-fiber meal, AUCo-isomin values were significantly higher compared to that following the ADA meal
after the high-fat meal, Cₘₐₓ values were significantly lower compared to that following ADA meal
high-fiber meal improved IN-105 absorption, while high-fat meal, though it lowered the peak levels, did not affect total absorption.

### Example 4: Safety Results

All randomized patients who received at least one dose of study drug were included in the safety evaluation. Patients were analyzed according to treatment sequence. All available safety data collected up to the end of the study (i.e. through the last follow-up evaluation) were included in the safety analysis. IN-105 and IN-105 placebo administered under various dosing conditions (e.g., pre-meal times of 10, 20, or 30 minutes; 4 to 6 hours between meal times; and with high-fat or high-fiber meals) were safe and well tolerated in T2DM patients.

Amongst total 51 patients exposed to IN-105, 25 patients (6, 6 and 13 in Cohorts 1, 2 and 3 respectively) reported 66 TEAEs [10, 13 and 43 in Cohorts 1, 2 and 3 respectively]. Majority of TEAEs were mild in severity and 5 TEAEs were severe (all Hypoglycaemia; 2 each in Cohort-1 and -2, and 1 in Cohort-3). No deaths or serious adverse events (SAEs) occurred. There were no discontinuations due to AEs or SAEs. No other clinically significant abnormal findings were observed for the other laboratory parameters, physical examination, vital signs, or ECG data. There was lowering of hematocrit and hemoglobin values by end of study, which may possibly be attributed to study-related blood loss. There was no consumption of concomitant medications that interfered with study treatment.

There were 43 events of hypoglycemia in 15 patients; 41 hypoglycemia events were related to metformin, IN 105, or both. All of them resolved without treatment except one, which required treatment with glucose tablets. Of the 43 events of hypoglycemia, 41 events occurred within 2 hours of IN-105 administration. Generally, the duration of hypoglycemia symptoms was approximately 30 minutes.

### Example 5: Hypoglycaemic Adverse Events

There were 43 events of Hypoglycaemia in 15 patients (3 patients each in Cohort-1 and -2, and 9 in Cohort-3); 41 Hypoglycaemia events were related to metformin, IN-105, or both. Except for 2 events, all other hypoglycaemic events were asymptomatic and detected by glucose measurements. There were no discontinuations due to Hypoglycaemia. Majority of hypoglycaemic events were mild (did not interfere with patient's usual function, 83.7%) in severity. Of the 43 events, 41 hypoglycaemic events occurred within 2 hours of IN-105 administration and 2 events occurred closer to 6 and 46 hours after IN-105 administration.

In Cohort-1, there were 4 events of Hypoglycaemia (3 in 30 minute pre-meal dosing time group and 1 in 20 minute group).

In Cohort-2, there were 5 events of Hypoglycaemia (1 in 4 hour, 2 each in 5 and 6 hour treatment groups); all were observed in the afternoon—4 occurring after and 1 before the administration of second dose of IN-105.

In Cohort-3, when IN-105 was administered twice daily, 34 hypoglycaemic events occurred; 13 events occurred in the morning while the remaining 21 Hypoglycaemia events occurred during afternoon and evening. When IN-105 was administered only during the morning, 9 events were observed, of which 8 occurred during the morning.

Generally, the duration of hypoglycaemic symptoms was for approximately 30 minutes. All the hypoglycaemic episodes resolved without treatment except one (1 patient in Cohort-1 [51 mg/dL] who was administered IN-105, 20 min before food) that required treatment with glucose tablets.

### Result and Discussion

Maximum absorption of IN-105 was noted with pre-meal dosing time of 30 minutes. However, maximal peak concentration of IN-105 was already attained before the initiation of the meal, cf. FIG. 2a, resulting in a low glucose lowering potential in the post-meal period. A better control of prandial glucose (PPG) levels, resulting in a lower divergence from baseline was observed with pre-meal dosing time of 10 minutes. Additionally, in terms of the GM ratio, pre-meal dosing time of 20 minutes demonstrated better glucose lowering activity compared to the 30 minutes group. Thus, a pre-meal dosing time of 10 to 20 minutes has been considered as optimal for postprandial IN-105 administration, closer to the other prandial insulins currently in use e.g. 5-10 min prior to meal for insulin aspart.

Elevated postprandial glucose level is an important contributor to overall hyperglycaemia in diabetes. Following meal ingestion in healthy individuals, physiological insulin levels in blood reach half of the maximal concentration in approximately 16-18 minutes and peak within 30-45 minutes. This first phase of insulin secretion is deficient in patients with T2DM.

In this study, the plasma glucose lowering effect of IN-105 was observed 16-37 minutes post-dose in patients with T2DM mimicking physiological insulin. This rapid-onset profile of IN-105 results in effective lowering of glucose exposure early in the post-meal period, thereby minimizing chances of postprandial Hypoglycaemia. The duration of glucose lowering action of IN-105 of 2-3 hours helps in decreasing the number of hypoglycaemic incidences and is found to be shorter in comparison to other fast acting insulins (3-5 hours). Reduced risk of hypoglycaemia and convenience of oral delivery may help to improve patient compliance with insulin therapy, considered as an important factor for achieving HbAlc targets.

The IN-105 AUC₀₋ᵢₛₒₘᵢₙ and Cₘₐₓ was lower for the afternoon dose compared to morning dose in the group with 4 and 5 hours between-meal-interval; while there was no difference in the group with 6 hours between-meal interval. Although IN-105 exposure (plasma AUC) showed a progressive increase through 4, 5 and 6 hours of between-meal interval, the glucose-lowering response, after 4, 5 and 6 hours was similar. Thus, between-meal dosing interval had an impact on the IN-105 PK, but it did not translate into significant impact on the PD parameters. The reason for similar PD with different PK may be due to persistent blood glucose levels from the morning meal in all the groups and less likely due to impact on insulin absorption. Additionally, even though high intra-subject variability for PK parameters was observed, intra-subject variability for the baseline-adjusted PD parameters (AUC₀₋ᵢₛₒₘᵢₙ and Cₘᵢₙ) was not different from placebo and thus not considered to be of significant concern.

High-fat and high fiber meal decreased IN-105 absorption (as compared to ADA-meal) in morning. Additionally, high-fiber meal in morning increased absorption of the subsequent (afternoon) dose of IN-105. IN-105 reached effective concentrations in blood even in the presence of food of varying composition and the PD effect was retained. Morning high-fat and high-fiber meals resulted in lower glucose in the morning as well as afternoon in comparison to morning ADA-meal. However, the lowest glucose concentration was observed in the ADA-meal group in the morning and in the high-fiber meal group in the afternoon. Overall, although there was a difference in PK of accompanying as well as subsequent dose of IN-105 (insulin tregopil) observed with different types of meal, this did not translate into a consistently significant impact on the PD parameters. A probable explanation for the variance of plasma PK levels and PD effects is probably because IN-105 levels in the portal circulation are much more determinative of metabolic effects than peripheral levels. Hepatic glucose production suppression probably reflects a more insulinized liver resulting from hepato preferential availability of IN-105. In present invention, most of the hypoglycaemia cases reported were mild to moderate in intensity and resolved without treatment, thus, acting as an early indicator of better safety.

1. A method of controlling the postprandial blood glucose level in a subject, the method comprising:
orally administering to a subject an orally administrable oligoethylene glycol conjugate of insulin, and wherein orally administering is carried out during a time period from about 5 to 25 minutes prior to a meal.

2. (canceled)

3. The method of claim 1, wherein the oligoethylene glycol conjugate of insulin comprises insulin linked at amino acid 29 of the insulin B chain via an amide bond to a moiety of the formula 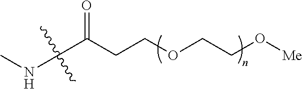
wherein n is an integer from 2 to 5.

4. The method of claim 3, wherein n is the integer 3.

5. The method of claim 3 wherein oligoethylene glycol conjugate is administered within a time period from about 10 to 20 minutes prior to a meal.

6. The method of any of claim 3 wherein the oligoethylene glycol conjugate comprises human insulin.

7. (canceled)

8. The method of claim 4, wherein the oligoethylene glycol conjugate is IN-105.

9. The method of claim 8, wherein the oligoethylene glycol conjugate is administered at a dose in the range of 10 to 60 mg.

10. The method of claim 9, wherein the oligoethylene glycol conjugate is administered at a dose of about 30 mg.

11. The method claim 1, wherein the subject is a human.

12. The method of claim 1, wherein the subject is suffering from Diabetes mellitus.

13. The method of claim 12, wherein the Diabetes mellitus is Diabetes mellitus type 2.

14. The method of claim 1, wherein orally administering is carried out once during said time period prior to a meal.

15. (canceled)

16. A method of controlling the postprandial blood glucose level in a subject, the method comprising:
orally administering to a human subject an orally administrable oligoethylene glycol conjugate of insulin, wherein orally administering is carried out during a time period from about 10 to 20 minutes prior to a meal, and is administered at a dose in the range of 10 to 60 mg, and wherein the wherein the oligoethylene glycol conjugate of insulin comprises insulin linked at amino acid 29 of the insulin B chain via an amide bond to a moiety of the formula 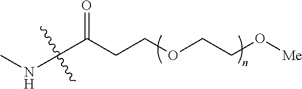
wherein n is 3.
